(19) United States
(12) Patent Application Publication
(10) Pub. No.: US 2021/0308104 A1
(43) Pub. Date: Oct. 7, 2021

(54) PHARMACEUTICAL COMPOSITION COMPRISING ELTROMBOPAG OLAMINE

(71) Applicant: SANOVEL ILAC SANAYI VE TICARET ANONIM SIRKETI, Istanbul (TR)

(72) Inventors: ALI TURKYILMAZ, Istanbul (TR); EDIZ YILDIRIM, Istanbul (TR); GULCIN TOK, Istanbul (TR)

(73) Assignee: SANOVEL ILAC SANAYI VE TICARET ANONIM SIRKETI, Istanbul (TR)

(21) Appl. No.: 17/263,856

(22) PCT Filed: Jul. 26, 2019
(86) PCT No.: PCT/TR2019/050626

§ 371 Date: Jan. 27, 2021

(30) Foreign Application Priority Data
Aug. 2, 2018 (TR) 2018/11214

(51) Int. Cl. A61K 31/4152; A61K 9/48

(52) U.S. Cl. CPC A61K 31/4152; A61K 9/48; A61K 9/485; A61K 9/4866; A61K 9/4858

(57) ABSTRACT

The present invention relates to a pharmaceutical capsule composition comprising eltrombopag olamine or a pharmaceutically acceptable salt thereof for use in the treatment of thrombocytopenia. Furthermore, the present invention relates to an improved, simple, rapid, cost-effective, time-saving and industrially available method of preparing the capsule composition comprising eltrombopag olamine.

### FIELD OF THE INVENTION

The present invention relates to a pharmaceutical capsule composition comprising eltrombopag olamine or a pharmaceutically acceptable salt thereof for use in the treatment of thrombocytopenia. Furthermore, the present invention relates to an improved, simple, rapid, cost-effective, time-saving and industrially available method of preparing the capsule composition comprising eltrombopag olamine.

### BACKGROUND OF THE INVENTION

Thrombopoietin (THPO), also known as megakaryocyte growth and development factor (MGDF), is a protein that in humans is encoded by the THPO gene.

The thrombopoietin receptor agonists mimic the action of thrombopoietin on its receptor and stimulate the activation, proliferation and maturation of megakaryocytes, resulting in an increase in circulating platelet counts. Thrombopoietin itself acts in this manner, but when recombinant thrombopoietins were used clinically, they were found to cause rebound thrombocytopenia, probably due to induction of anti-thrombopoietin antibodies. For this reason, direct administration of thrombopoietin was abandoned as an approach to treating thrombocytopenia and other approaches for activating the thrombopoietin receptor were sought.

Two thrombopoietin receptor agonists were subsequently developed and are now in clinical use for chronic idiopathic thrombocytopenic purpura (ITP) and for other thrombocytopenic conditions.

Eltrombopag olamine is a peptide-like, small molecular weight agonist of the thrombopoietin receptor. This agent is given by mouth and result in significant increases in platelet counts in normal persons as well as patients with idiopathic thrombocytopenic purpura (ITP).

Eltrombopag olamine is a small molecular weight peptide-like molecule that binds to the transmembrane domain of the thrombopoietin receptor and causes its activation and the proliferation and differentiation of megakaryocytes, with a resultant increase in synthesis and release of platelets. In multiple clinical trials, eltrombopag olamine was shown to raise the platelet count in patients with idiopathic thrombocytopenic purpura (ITP), aplastic anemia and cirrhosis due to chronic hepatitis C during interferon therapy. Eltrombopag olamine was approved for use in the United States in 2008 for the treatment of ITP and its indications have subsequently been expanded to other thrombocytopenic conditions.

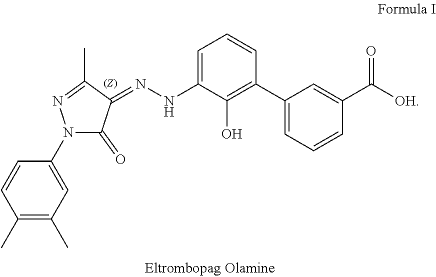 [SMILES: CC1=NN(c2ccc(C)c(C)c2)C(=O)/C1=N\Nc1cccc(-c2cccc(C(=O)O)c2)c1O]

In the state of art, the application EP1889838B1 is the molecule patent of eltrombopag olamine. Another application EP3041511A2 discloses pharmaceutical compounds of eltrombopag olamine in the form of tablets and the methods for their preparation.

In the present art, eltrombopag olamine is used in tablet form and individually adjusted according to the patient's platelet count to increase the patient's platelet count. However, an acceptable tablet dosage form is difficult to formulate on a commercial scale. The formula and the manufacturing process should be in a tablet form that maintains its integrity until use, while having acceptable dissolution and disintegration properties to provide the desired profile in use. However, there are difficulties in the preparation of high-quality tablet forms as well as in achieving the desired dissolution rate, stability and a long shelf-life, since the pharmaceutically active compounds that can react with the excipients with low-solubility and/or commonly used excipients and the physical properties of the drug affect the properties of the tablet form.

### DETAILED DESCRIPTION OF THE INVENTION

The main object of the present invention is to provide a novel pharmaceutical capsule composition comprising eltrombopag olamine and at least one pharmaceutically acceptable excipient that eliminate the above-mentioned problems and provide additional advantages to the relevant prior art.

A further object of the present invention is to provide a solid oral form which eliminates the negative properties of the tablet form of eltrombopag olamine formulation in the prior art. Eltrompobag olamine in the capsule form instead of the tablet form as used in the prior art enhances the bioavailability by providing rapidly dissolving capsule walls and low flocculation during preparation.

Another object of the invention is to use hard capsule or hydroxypropyl methylcellulose capsules instead of soft gelatin capsules. Hydroxypropyl methylcellulose capsules and hard capsules are intended to dissolve eltrombopag olamine, the active substance, and increase the bioavailability of the total capsule composition.

Another object of the present invention is to provide a pharmaceutical composition to increase the circulating platelet counts by enhancing the activation of thrombopoietin receptor agonists. The formulator should be balance the unique properties of the drug with the properties of each excipient in order to prepare a solid dosage form that is safe, effective and easy to use.

Another object of the present invention is to provide a more stable capsule form with the desired dissolution and a long shelf life by selecting a specific particle size range.

A further object of the present invention is to provide a simple, fast, cost-effective, time-saving and industrially suitable method for the preparation of the capsule composition comprising eltrombopag olamine.

Detailed properties of the present invention are provided here in accordance with the objects summarized above.

The present invention relates to a pharmaceutical capsule composition comprising the active ingredient eltrombopag olamine or a pharmaceutically acceptable salt thereof and at least one pharmaceutically acceptable excipient.

In the pharmaceutical capsule composition comprising the eltrombopag olamine of the present invention, the particle size seems to play an active role in increasing the solubility of the active substance eltrombopag olamine and providing an appropriate bioavailability.

The term “particle size” refers to the cumulative volume size distribution tested by any conventionally recognised method, such as a laser diffraction method. The term d (_{0.1}) means the size at which 10% by volume of the particles are finer and the term d (_{0.5}) means the size at which 50% by volume of the particles are finer and the term d (_{0.9}) means the size at which 90% by volume of the particles are finer.

According to an embodiment of the present invention, the pharmaceutical capsule composition comprises eltrombopag olamine as the active substance, wherein the particle size (_{0.1}) of the eltrombopag olamine is less than 20 μm.

According to an embodiment of the present invention, the particle size d (_{0.1}) of eltrombopag olamine is preferably between 10 μm and 1 μm, most preferably between 8 μm and 1 μm.

According to an embodiment of the present invention, the pharmaceutical capsule composition comprises eltrombopag olamine as the active substance, wherein the particle size (_{0.5}) of the eltrombopag olamine is less than 50 μm.

According to an embodiment of the present invention, the particle size d (₀₅) of eltrombopag olamine is preferably between 40 μm and 10 μm, most preferably between 30 μm and 10 μm.

Particularly, the particle size distribution of D (_{0.5}) overcame the problems encountered in the prior art by increasing the solubility of the active substance eltrombopag olamine, resulting in enhanced bioavailability of the capsule composition.

According to an embodiment of the present invention, the pharmaceutical capsule composition comprises eltrombopag olamine as the active substance, wherein the particle size (_{0.9}) of the eltrombopag olamine is less than 100 μm.

According to an embodiment of the present invention, the particle size d (_{0.9}) of eltrombopag olamine is preferably between 80 μm and 30 μm, most preferably between 60 μm and 30 μm.

An embodiment of the present invention provides a pharmaceutical capsule composition comprising eltrombopag olamine and at least one pharmaceutically acceptable excipient that eliminate the above-mentioned problems and provide additional advantages to the relevant prior art, wherein d (_{0.1}) particle size is between 20 μm and 1 μm, d (_{0.5}) particle size is between 50 μm and 10 μm and d (_{0.9}) particle size is between 100 μm and 30 μm.

According to the present invention, the amount of eltrombopag olamine is 1% to 60.0% by weight of the total formulation.

In an embodiment of the present invention, eltrombopag olamine is present preferably between 5.0-55.0%, most preferably between 5.0-45.0% by weight of the total formulation.

The pharmaceutical capsule composition comprises, more specifically, eltrombopag olamine or a pharmaceutically acceptable salt thereof and at least one excipient selected from the group comprising at least one filler, at least one binder, at least one binder, at least one glidant, at least one lubricant.

The pharmaceutical capsule composition of the present invention comprises at least one filler.

The filler that is present in the pharmaceutical capsule composition comprises at least one agent or a combination of two or more agents selected from microcrystalline cellulose, kaolin, cellulose (eg, microcrystalline cellulose, powdered cellulose), pre-gelatinized starch, starch, lactitol, mannitol, sorbitol, maltodextrin, powdered sugar, compressible sugar, sucrose, dextrose and inositol.

The mentioned pharmaceutical capsule composition preferably comprises microcrystalline cellulose and/or mannitol and/or sucrose or a mixture thereof as a filler.

The amount of filler is present in the range of 5.0% to 90.0% by weight of the total formulation.

In an embodiment of the present invention, the filler is present preferably between 5.0-80.0%, most preferably between 5.0-70.0% by weight of the total formulation.

In the current state of art, the use of mannitol contributes to maintaining the stability in the capsule form, particularly by balancing the moisture-sensitive active ingredient eltrombopag olamine with its non-hygroscopic property.

In another aspect, the compound present in pharmaceutical capsule compositions has the tendency to form insoluble metal complexes when contacted with excipients comprising coordinate metal, dissolve slowly from solid dosage forms, and to undergo Maillard reaction when contacted with excipients comprising reducing sugars. Mannitol as a filler does not wear out from cold, dilute acids, alkali or atmospheric oxygen in the absence of catalyst. Thus, mannitol does not undergo Maillard reactions, reducing the negative properties of eltrombopag olamine on the patient.

The pharmaceutical capsule composition of the present invention comprises at least one disintegrant.

The disintegrant that is present in the mentioned pharmaceutical capsule composition comprises at least one agent or a combination of two or more agents selected from croscarmellose sodium, sodium carbonate, hydroxypropyl cellulose (HPC), mycrocrystalline cellulose, cross-linked polyvinylpyrrolidone (crospovidone), copovidone, polycarbophil, low substituted poloxamer, sodium starch glycolate, starch, pregelatinized starch, alginic acid and alginates, ion exchange resins, sodium carboxy methyl cellulose, docusate sodium, guar gum, sodium alginate, sodium glycine carbonate, polysorbate.

The pharmaceutical capsule composition preferably comprises croscarmellose sodium as disintegrant.

The amount of disintegrant is present in the range of 3.0% to 50.0% by weight of the total formulation.

In an embodiment of the present invention, the disintegrant is present preferably between 3.0-45.0%, most preferably between 3.0-40.0% by weight of the total formulation.

It was also seen that the amount of active ingredient, disintegrant and filler in the composition is significantly important in order to obtain capsules that provide both high solubility and stability.

In the mentioned pharmaceutical capsule composition, the weight ratio of eltrombopag olamine to filler is between 0.01 and 12, preferably between 0.01 and 6, more preferably between 0.01 and 1.

In the mentioned pharmaceutical capsule composition, the weight ratio of eltrombopag olamine to disintegrant is between 0.02 and 20, preferably between 0.05 and 10, more preferably between 0.1 and 8.

The mentioned pharmaceutical capsule composition can comprise at least one binder.

The binder that is present in the mentioned pharmaceutical capsule composition comprises at least one agent or a combination of two or more agents selected from copovidone, copolyvidone, polyvinylpyrrolidone (PVP), povidone, carnauba wax, hydroxypropyl methyl cellulose (HPMC), pullulan, polymethacrylate, glyceryl behenate, hydroxypropyl cellulose (HPC), carboxy methyl cellulose (CMC), methyl cellulose (MC), hydroxyethyl cellulose, sodyum carboxymethyl cellulose (Na CMC), ethyl cellulose, microcrystalline cellulose, polymethacrylates, polyethylene oxide, polyvinyl alcohol, polycarbophil, polyvinil acetate and copolymers thereof, gelatin, starch, pregelatinized starch, xanthan gum, guar gum, alginate, carrageenan, collagen, agar, pectin, hyaluronic acid, carbomer, cellulose acetate phthalate, hydroxypropyl starch, hydroxyethyl methyl cellulose, poloxamer, polyethylene glycol (PEG), non-reducing sugars, natural gums, tragacanth, polyacrylamide, bentonite, laponite, cetostearyl alcohol, polyethylene-alkyl ethers, acacia gum, and polydextrose.

The binder that is present in the pharmaceutical capsule composition preferably comprises povidone, hydroxypropyl methylcellulose, polyethlene glycol.

The mentioned pharmaceutical capsule composition comprises at least one glidant.

The glidant that is present in the mentioned pharmaceutical capsule composition comprises at least one agent or a combination of two or more agents selected from talc, colloidal silicon dioxide, colloidal silica, starch.

The mentioned pharmaceutical capsule composition comprises at least one lubricant.

The lubricant that is present in the mentioned pharmaceutical capsule composition comprises at least one agent or a combination of two or more agents selected from calcium stearate, sodium stearyl fumarate, sodium lauryl sulfate, zinc stearate, magnesium sterate, mineral oil, talc, polyethylene glycol, glyceryl monostearate, glyceryl palmitostearate, magnesium lauryl sulfate, fumaric acid, stearic acid, hydrogenated natural oils, silica, and paraffin.

The pharmaceutical capsule comprises lubricant which is preferably magnesium stearate and/or sodium stearyl fumarate and/or talc, or a mixture thereof.

### Example 1: Eltrombopag Olamine Capsule

Total capsule formulation comprises;
a. 1.0-60.0% by weight of eltrombopag olamin
b. 5.0-90.0% by weight of filler
c. 0.1-40.0% by weight of binder
d. 3.0-50.0% by weight of disintegrant
e. 0.1-6.0% by weight of glidant
f. 0.1-10% by weight of lubricant

### Example 2: Eltrombopag Olamine Capsule Formulation

Total capsule formulation comprises;
a. 1.0-60.0% by weight of eltrombopag olamine
b. 3.0-20.0% by weight of microcrystalline cellulose
c. 2.0-30.0% by weight of mannitol
d. 0.1-20.0% by weight of povidone
e. 3.0-50.0% by weight of croscarmellose sodium
f. 1.0-40.0% by weight of sucrose
g. 0.1-6.0% by weight of colloidal silicon dioxide
h. 0.1-10.0 by weight of magnesium stearate.

### Manufacturing Process;

Sieving individually eltrombopag olamine, microcrystalline cellulose, mannitol, povidone (preferably povidone K-30) and croscarmellose sodium by an appropriate mesh, then mixing and obtaining wet granulation with water. Subjecting the wet granule to wet sieving, drying on fluidized bed and sieving the dried granules by an appropriate mesh to obtain a homogenous powder Sieving croscarmellose sodium, sucrose, colloidal silicon dioxide by an appropriate mesh and adding them to inner phase and mixing for 15 minutes.

### Example 3: Eltrombopag Olamine Capsule Formulation

Total capsule formulation comprises;
a. 1.0-60.0% by weight of eltrombopag olamine
b. 3.0-60.0% by weight of microcrystalline cellulose
c. 2.0-30.0% by weight of mannitol
d. 3.0-50.0% by weight of croscarmellose sodium
e. 0.1-6.0% by weight of colloidal silicon dioxide
f. 0.1-10.0% by weight of magnesium stearate.

### Manufacturing Process;

Sieving individually eltrombopag olamine, ⅓ of microcrystalline cellulose and mannitol by an appropriate mesh, then mixing and obtaining wet granulation with water. Subjecting the wet granule to wet sieving, drying on fluidized bed and sieving the dried granules by an appropriate mesh to obtain a homogenous powder Sieving ⅔ of microcrystalline cellulose, croscarmellose sodium and colloidal silicon dioxide by an appropriate mesh and adding them to inner phase and mixing for 15 minutes. Finally, sieving magnesium stearate by an appropriate mesh and adding it to the mixture and mixing for another 5 minutes.

### Example 4: Eltrombopag Olamine Capsule Formulation

Total capsule formulation comprises;
a. 1.0-60.0% by weight of eltrombopag olamine
b. 3.0-50.0% by weight of microcrystalline cellulose
c. 2.0-40.0% by weight of mannitol
d. 1.0-40.0% by weight of hydroxypropyl methyl cellulose
e. 3.0-50.0% by weight of croscarmellose sodium
f. 0.1-6.0% by weight of colloidal silicon dioxide
g. 0.1-4.0% by weight of talc
h. 0.1-6.0% by weight of sodium stearyl fumarate.

### Manufacturing Process;

Sieving individually eltrombopag olamine, ⅓ of microcrystalline cellulose, mannitol and ⅓ of HPMC by an appropriate mesh, then mixing and obtaining wet granulation with water. Subjecting the wet granule to wet sieving, drying on fluidized bed and sieving the dried granules by an appropriate mesh to obtain an homogenous powder Sieving ⅔ of microcrystalline cellulose, ⅔ of HPMC, croscarmellose sodium and colloidal silicon dioxide by an appropriate mesh and adding them to inner phase and mixing for 15 minutes. Finally, sieving talc and sodium stearyl fumarate by an appropriate mesh and adding it to the mixture and mixing for another 5 minutes.

### Example 5: Eltrombopag Olamine Capsule Formulation

Total capsule formulation comprises;
a. 1.0-60.0% by weight of eltrombopag olamine
b. 3.0-50.0% by weight of microcrystalline cellulose
c. 2.0-40.0% by weight of mannitol
d. 1.0-40.0% polyethylene glycol
e. 3.0-50.0% by weight of croscarmellose sodium
f. 0.1-6.0% by weight of colloidal silicon dioxide
g. 0.1-4.0% by weight of talc
h. 0.1-6.0% by weight of sodium stearyl fumarate.
   Manufacturing process;

Sieving individually eltrombopag olamine, ⅓ of microcrystalline cellulose, mannitol and ⅓ of polyethylene glycol by an appropriate mesh, then mixing and obtaining wet granulation with water. Subjecting the wet granule to wet sieving, drying on fluidized bed and sieving the dried granules by an appropriate mesh to obtain a homogenous powder Sieving ⅔ of microcrystalline cellulose, ⅔ of polyethylene glycol, croscarmellose sodium and colloidal silicon dioxide by an appropriate mesh and adding them to inner phase and mixing for 15 minutes. Finally, sieving talc and sodium stearyl fumarate by an appropriate mesh and adding it to the mixture and mixing for another 5 minutes.

### Example 6: Eltrombopag Olamine Capsule Formulation

Total capsule formulation comprises;
a. 1.0-60.0% by weight of eltrombopag olamine
b. 3.0-50.0% by weight of microcrystalline cellulose
c. 2.0-40.0% by weight of mannitol
d. 1.0-40.0% by weight of povidone
e. 3.0-50.0% by weight of croscarmellose sodium
f. 0.1-6.0% by weight of colloidal silicon dioxide
g. 0.1-4.0% by weight of talc
h. 0.1-6.0% by weight of sodium stearyl fumarate.

### Manufacturing Process;

Sieving individually eltrombopag olamine, ⅓ of microcrystalline cellulose, mannitol and ⅓ of povidone by an appropriate mesh, then mixing and obtaining wet granulation with water. Subjecting the wet granule to wet sieving, drying on fluidized bed and sieving the dried granules by an appropriate mesh to obtain a homogenous powder Sieving ⅔ of microcrystalline cellulose, ⅔ of povidone, croscarmellose sodium and colloidal silicon dioxide by an appropriate mesh and adding them to inner phase and mixing for 15 minutes. Finally, sieving talc and sodium stearyl fumarate by an appropriate mesh and adding it to the mixture and mixing for another 5 minutes.

1. A pharmaceutical capsule composition, comprising eltrombopag olamine or a pharmaceutically acceptable salt thereof and at least one pharmaceutically acceptable excipient.

2. The pharmaceutical capsule composition according to claim 1, wherein the amount of eltrombopag olamine is in the range of 1.0 to 60.0% by weight of the total formulation.

3. The pharmaceutical capsule composition according to claim 1, wherein D(0.5) particle size distribution of eltrombopag olamine is less than 50 μm.

4. The pharmaceutical capsule composition according to claim 1, wherein at least one of the pharmaceutically acceptable excipients is a filler.

5. The pharmaceutical capsule composition according to claim 4, wherein the filler comprises at least one agent or a combination of two or more agents selected from microcrystalline cellulose, kaolin, cellulose (eg, microcrystalline cellulose, powdered cellulose), pre-gelatinized starch, starch, lactitol, mannitol, sorbitol, maltodextrin, powdered sugar, compressible sugar, sucrose, dextrose and inositol.

6. The pharmaceutical capsule composition according to claim 5, wherein the composition comprising filler, which is preferably selected from microcrystalline cellulose, mannitol, sucrose or a mixture thereof.

7. The pharmaceutical capsule composition according to claim 2, wherein the ratio of the eltrombopag olamine to the filler is in the range of 0.01 to 12.0, preferably in the range 0.01 to 6.0, most preferably in the range of 0.01 to 1.0 by weight of the total capsule.

8. The pharmaceutical capsule composition according to claim 1, wherein at least one of the pharmaceutically acceptable excipients is a disintegrant.

9. The pharmaceutical capsule composition according to claim 8, wherein the disintegrant that is present in the mentioned pharmaceutical capsule composition comprises at least one agent or a combination of two or more agents selected from croscarmellose sodium, sodium carbonate, hydroxypropyl cellulose (HPC), microcrystalline cellulose, cross-linked polyvinylpyrrolidone (crospovidone), copovidone, polycarbophil, low substituted poloxamer, sodium starch glycolate, starch, pregelatinized starch, alginic acid and alginates, ion exchange resins, sodium carboxy methyl cellulose, docusate sodium, guar gum, sodium alginate, sodium glycine carbonate, polysorbate.

10. The pharmaceutical capsule composition according to claim 2, wherein the weight ratio of eltrombopag olamine to disintegrant is between 0.02 and 20.0, preferably between 0.05 and 10.0, more preferably between 0.1 and 8.0 by weight of the total capsule.

11. The pharmaceutical capsule composition according to claim 1, wherein the total formulation comprises
a. 1.0-60.0% by weight of eltrombopag olamine
b. 3.0-20.0% by weight of microcrystalline cellulose
c. 2.0-30.0% by weight of mannitol
d. 0.1-20.0% by weight of povidone
e. 3.0-50.0% by weight of croscarmellose sodium
f. 1.0-40.0% by weight of sucrose
g. 0.1-6.0% by weight of colloidal silicon dioxide
h. 0.1-10.0% by weight of magnesium stearate

12. The pharmaceutical capsule composition according to claim 1, wherein the total formulation comprises
a. 1.0-60.0% by weight of eltrombopag olamine
b. 3.0-50.0% by weight of microcrystalline cellulose
c. 2.0-40.0% by weight of mannitol
d. 3.0-50.0% by weight of croscarmellose sodium
e. 0.1-6.0% by weight of colloidal silicon dioxide
f. 0.1-10.0% by weight of magnesium stearate

13. The pharmaceutical capsule composition according to claim 1, wherein the total formulation comprises
a. 1.0-60.0% by weight of eltrombopag olamine
b. 3.0-50.0% by weight of microcrystalline cellulose
c. 2.0-40.0% by weight of mannitol
d. 1.0-40.0% by weight of hydroxypropyl methyl cellulose
e. 3.0-50.0% by weight of croscarmellose sodium
f. 0.1-6.0% by weight of colloidal silicon dioxide
g. 0.1-4.0% by weight of talc
h. 0.1-6.0% by weight of sodium stearyl fumarate.

14. The pharmaceutical capsule composition according claim 1, wherein the total formulation comprises
a. 1.0-60.0% by weight of eltrombopag olamine
b. 3.0-50.0% by weight of microcrystalline cellulose
c. 2.0-40.0% by weight of mannitol
d. 1.0-40.0% polyethylene glycol
e. 3.0-50.0% by weight of croscarmellose sodium
f. 0.1-6.0% by weight of colloidal silicon dioxide
g. 0.1-4.0% by weight of talc
h. 0.1-6.0% by weight of sodium stearyl fumarate.

15. The pharmaceutical capsule composition according to claim 1, wherein the total formulation comprises
a. 1.0-60.0% by weight of eltrombopag olamine
b. 3.0-50.0% by weight of microcrystalline cellulose
c. 2.0-40.0% by weight of mannitol
d. 1.0-40.0% by weight of povidone
e. 3.0-50.0% by weight of croscarmellose sodium
f. 0.1-6.0% by weight of colloidal silicon dioxide
g. 0.1-4.0% by weight of talc
h. 0.1-6.0% by weight of sodium stearyl fumarate.
